(19) United States
(12) Patent Application Publication
(10) Pub. No.: US 2010/0324215 A1
(43) Pub. Date: Dec. 23, 2010

(54) AFFINITY STRUCTURES FOR THE SPECIFIC BINDING OF SUBSTANCES BY MEANS OF NON-COVALENT INTERACTION TYPES

(75) Inventors: Heiko Andresen, Berlin (DE); Frank Bier, Potsdam (DE); Martin Katterle, Falkensee (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: Fraunhofer-Gesellschaft zur Forderung der angewandten Forschung e.V., Munchen (DE)

(21) Appl. No.: 12/743,385

(22) PCT Filed: Nov. 25, 2008
(86) PCT No.: PCT/EP2008/009984

§ 371 Date: May 18, 2010

(30) Foreign Application Priority Data
Nov. 26, 2007 (DE) 10 2007 056 875.6

(51) Int. Cl. C07K 2/00; C08G 69/48; C08G 63/91; C08G 71/02; C08G 59/14; C07K 14/00; C07K 14/42; C07H 1/00; C07H 21/00

(52) U.S. Cl. 525/54.1; 525/50; 525/54.2; 525/418; 525/420; 525/453; 525/523; 525/540; 530/300; 530/350; 530/370; 536/1.11; 536/22.1

(57) ABSTRACT

A method for producing affinity binders having affinitive or highly affinitive binding structures for non-covalent interaction types includes a) contacting a target substance with at least one ligand which is capable of non-covalently binding to the target substance for a sufficient time that the at least one ligand can specifically attach to target regions of the target substance and form a ligand-target substance complex; b) embedding the complex in a structure-providing component and fixing by binding the ligand(s) of the complex to the structure-providing component; and c) removing the target substance such that a structure with a defined spatial arrangement of the ligand(s) is formed which has capacity to specifically bind the respective target substance again.

### RELATED APPLICATIONS

This is a §371 of International Application No. PCT/EP2008/009984, with an international filing date of Nov. 25, 2008 (WO 2009/068244 A1, published Jun. 4, 2009), which is based on German Patent Application No. 10 2007 056 875.6, filed Nov. 26, 2007, the subject matter of which is incorporated by reference.

### TECHNICAL FIELD

This disclosure relates to affinity structures, particularly to affinity binders having affinitive binding structures for non-covalent interaction types and methods for producing affinity binders having affinitive binding structures for non-covalent interaction types.

### BACKGROUND

Reagents with the capacity for the specific binding of substances by means of non-covalent interaction types (affinity binders) find many uses in the identification of biomolecules and non-biological substances. In medicine, such reagents form the basis of modern diagnostic methods. In pharmaceutics, they are used as active ingredients. Affinity binders are furthermore the basis of many analytical methods, in particular in biomedical research as well as separation and cleaning methods in technical processes.

Affinitive identification and binding form the basis of numerous cellular processes in biological systems. Examples are protein-protein interactions within the scope of signal pathways and regulatory processes or the binding and neutralization of pathogens, toxins or other foreign substances by means of specific antibodies as a result of the immune response. Antibodies are a highly variable class of proteins. In the course of the immune response, antibodies to any substances foreign to the organism can in principle be developed naturally and be selected. The variability relies on a mechanism which results in a random combination of amino acids in the antibody binding domain—the paratope. In this way, binders with a unique specificity and affinity are formed. Furthermore, antibodies which react specifically with a particular substance can be produced specifically by methodically bringing a (host) organism into contact with this substance (immunizing it). In special methods, antibody molecules having a singular structure and specificity can be produced in this way, so-called monoclonal antibodies (Köhler and Milstein 1975, 1984 Nobel Prize in Medicine).

Non-covalent bonds between substances are based on physicochemical interaction types, e.g., hydrogen bonds, ionic and hydrophobic interactions and van der Waals forces, as well as their spatial arrangement and induced fit.

In particular in protein-protein interactions, for example, the bond between an anti-body and a protein antigen, the physicochemical interactions are often determined by short, continuous amino acid sequences within the molecule (peptides). This applies to both the binding domain of the antibody (paratope, e.g., Laune et al., 2002) and that of the protein (epitope, e.g., Andresen et al., 2006). For this reason, the antibodies and proteins in these types of molecular interactions can be substituted with functional peptidic binders. These often have identical or only slightly changed binding properties in terms of specificity and affinity as the original molecules.

Other biochemical interaction types in turn can be attributed to the physicochemical interaction of carbohydrates with other molecules (proteins, peptides, carbohydrates). Alternatively, specific binder molecules can be produced artificially, for example, as peptide mimetics or nucleic acid aptamers (Kleinjung et al., 1998).

Molecularly imprinted polymers (MIPs) represent one category of completely synthetic reagents for the binding of molecules. Molecular imprinting, also referred to as template method, includes the arrangement of polymerizable functional monomers around a print molecule. This is achieved by using non-covalent interactions or by reversible covalent interactions (covalent imprinting) between the print molecule and the functional monomers. The complexes thus obtained are then introduced into a highly cross-linked, macroporous polymer matrix by means of polymerization. An extraction of the print molecule leaves sites having a special shape and functional groups which are complementary to the original print molecule behind in the polymer (Mosbach et al., 1994, Wulff et al., 1993).

Despite the above-mentioned importance, the reagents for the binding of substances known in the prior art (e.g., antibodies, proteins, peptides, MIPs) still present certain drawbacks.

The production of antibodies is inevitably connected with animal experiments as the animals are needed as host organisms to form and produce antibodies. In special cases, it is furthermore not possible to produce antibodies as the substances are either toxic and result in the death of the animal or as the substances are broken down too quickly in the organism such that no effective immune response can be generated. The production of antibodies in general and monoclonal antibodies in particular and the production of proteins are furthermore associated with a high expenditure of time and costs. Proteins and antibodies are chemically and physically sensitive such that a reduction in binding capacity can occur under unfavorable environmental conditions (e.g. temperature, pH, humidity, chemical agents).

Just like simple carbohydrates or nucleic acid aptamers, peptidic binders can be produced synthetically in large amounts and at a low cost and are chemically and physically relatively stable in comparison to proteins/antibodies. These binders often have the disadvantage of an insufficient strength of the bond to the target substance due to a missing spatial structure of the binder which can support the binding process.

Molecularly imprinted polymers (MIPs) are chemically and physically stable and can be produced synthetically and thus at a low cost. The binding process of the MIPs relies first and foremost on the spatial induced fit of the target substance in the binding pocket of the polymer. For this reason, these binders often lack the specificity and affinity of the physicochemical interaction types of biomolecular components.

Accordingly, it could be helpful to provide new, improved reagents having the capacity for the identification and binding of substances by non-covalent interaction types (affinity binders). Such affinity binders offer a breadth of usage options in medicine and diagnostics, analytics and bioanalytics, pharmaceutics, in separation and cleaning methods, and can facilitate the production and handling in these fields as well as increase cost efficiency, versatility, stability, specificity and sensitivity of conventional methods. It could also be helpful to provide a particularly advantageous method for the production of such affinity binders.
Andresen, H., Zarse, K., Grötzinger, C., Hollidt, J., Ehrentreich-Förster, E., Bier, F. F., Kreuzer, O. J.: Development of peptide microarrays for epitope mapping of antibodies against the human TSH receptor. J. Immunol. Methods 315 (2006), 11-18.
Chardes, T., Villard, S., Ferrieres, G., Piechaczyk, M., Cerutti, M., Devauchelle, G., Pau, B.: Efficient amplification and direct sequencing of mouse variable regions from any immunoglobulin gene family. FEBS Lett. 452 (1999), 386.
Conrad, R. C., Baskerville, S., Ellington, A. D.: In vitro selection methodologies to probe RNA function and structure. Mol. Divers. 1 (1995), 69-78.
Kleinjung, F., Kluβmann, S., Erdmann, V. A., Scheller, F. W., Fürste, J. P., Bier, F. F.: High Affinity RNA as a Recognition Element in a Biosensor. Anal. Chem. 70 (1998), 328-331.
Köhler, G. & Milstein, C.: Continuous cultures of fused cells secreting antibody of predefined specificity. Nature 256 (1975), 495-497.
Laune, D., Molina, F., Ferrieres, G., Villard, S., Bes, C., Rieunier, F., Chardes, T., Granier, C.: Application of the Spot method to the identification of peptides and amino acids from the antibody paratope that contribute to antigen binding. J. Immunol. Methods 267 (2002), 53-70.
Mosbach, K.: Molecular imprinting. Trends Biochem. Sci. 19 (1994), 9-14.
Wulff, G.: The role of binding-site interactions in the molecular imprinting of polymers. Trends Biotechnol. 11 (1993), 85-87.

### SUMMARY

We provide a method for producing affinity binders having affinitive or highly affinitive binding structures for non-covalent interaction types, including a) contacting a target substance with at least one ligand which is capable of non-covalently binding to the target substance for a sufficient time that the at least one ligand can specifically attach to target regions of the target substance and form a ligand-target substance complex, b) embedding the complex in a structure-providing component and fixing by binding the ligand(s) of the complex to the structure-providing component, and c) removing the target substance such that a structure with a defined spatial arrangement of the ligand(s) is formed which has capacity to specifically bind the respective target substance again.

We also provide an affinity binder having affinitive or highly affinitive binding structures for non-covalent interaction types, obtained by the method for producing affinity binders and including a structure-providing component and at least one ligand integrated into the structure-providing component, wherein the structure-providing component holds the at least one ligand in a certain spatial arrangement which permits or affects the specific binding of the ligand(s) to a target substance.

### BRIEF DESCRIPTION OF THE DRAWINGS

FIG. 1 is a diagram of the generation of affinity binders by means of the combination of ligands and a structure-providing component, wherein: 1: target substance; 2: addition of one or more ligands which are provided with a polymerizable function (X); 3: attachment of the ligands to the target substance; 4: embedding of the complexes of target substance and peptides in the structure-providing component (polymer product of X); 5: cross-linking of the components with forming of a spatial structure in which the ligands are integrated fixedly and at defined positions; 6: removal of the template of the target substance from the structure.

FIG. 2 is a diagram of the affinity binder in which the binding process is combined with an effector function, wherein: 1: affinity binder in the original state capable to binding; 2: specific target substance; 3: binding process of the target substance results in changes within the affinity binder which represent an effector function or trigger the same.

### DETAILED DESCRIPTION

The method for the production of affinity binders having affinitive or highly affinitive binding structures for non-covalent interaction types comprises the following steps:
a) contacting a target substance with at least one ligand which is capable to non-covalently bind to the target substance for a sufficient time that the at least one ligand can specifically attach to the target regions of the target substance and forms a ligand-target substance complex;
b) embedding the complex in a structure-providing component and fixing by means of binding the ligand(s) of the complex to the structure-providing component; and
c) removing the target substance such that a structure with a defined spatial arrangement of the ligand(s) is formed which has the capacity to specifically bind the respective target substance again.

In a more specific example of the method, several ligands are fixed and integrated in the structure-providing component. These ligands can be identical and interact with recurring structural features (motifs) of the target substance. Alternatively, the ligands can also be different and interact with different motifs of the target substance.

An affinity binder which is obtainable by means of the above method comprises a structure-providing component, for example, a polymer molecularly imprinted by a certain target substance, into which at least one affinitive ligand for the target substance is integrated.

Ligands are herein molecules which have the capacity to specifically interact (i.e., selectively and with measurable affinity) with the same target substances by means of non-covalent interaction types, e.g., hydrogen bonds, ionic/hydrophobic interactions and van der Waals forces as well as “induced fit.” Some non-limiting examples of ligands are peptides or peptidomimetics, carbohydrates, e.g., (oligo)saccharides, nucleic acids, proteins and low-molecular weight organic substances.

By means of bioinformatical, combinatorial and/or molecular biological methods, affinity binders may be produced for almost any target substances.

The target substance is typically selected from the group including but not limited to a protein, peptide, carbohydrate, in particular an (oligo)saccharide, lectin, nucleic acid or another macromolecular unit, including a prokaryotic or eukaryotic cell, a virus or components thereof.

In one example, the target substance is a member of a known specific binding pair or a class of specific binding pairs, respectively. Some typical non-limiting examples of these are biotin/avidin, biotin/streptavidin, antibody/antigen, receptor/ligand, lectin/saccharide, DNA/DNA, RNA/RNA, DNA/RNA and the like. Further suitable examples are known to those skilled in the art or can easily be found in the literature.

The affinity binders require the provision of suitable ligands for the respective target substance.

The ligands are typically selected from the group including but not limited to a protein, peptide, carbohydrate, in particular an (oligo)saccharide, lectin, nucleic acid, low-molecular weight organic substance or mimetic of a protein ligand, peptide ligand, carbohydrate ligand or nucleic acid ligand. For example, the ligands may be selected from the known ligands for a certain target substance and thus represent the second member of a known specific binding pair.

Some non-limiting examples of ligands are antibodies or binding antibody fragments to one or more target proteins, e.g., native or genetically engineered Fab fragments or single-chain Fab derivatives for the affinitive binding of the corresponding antigen, e.g., tumor necrosis factor-alpha, peptides from the paratope of an antibody, e.g., to angiotensin II, lysozyme or the like, Fab fragments for the affinitive binding of virus particles or the like. Other, more general examples are monovalent or polyvalent aptamers for the binding of proteins, particles, cells or low-molecular weight substances (e.g., citrullin, flavin mononucleotide, neomycin B or the like).

A variety of such ligands against different target substances are already commercially available and usable.

However, it is also possible to develop and select new ligands for a target substance. The ligands can be derived by means of molecular biological methods from other molecules capable of binding (e.g., proteins, antibodies, carbohydrates, in particular saccharides) or also generated de novo. Alternatively, they can be determined by methods for the mapping (e.g., epitope mapping, paratope mapping) of known structures, in combinatorial methods or by computer-assisted modelling methods.

The selection can in the case of peptidic ligands, for example, take place by means of molecular biological testing and characterization of the paratope of monoclonal antibodies or of the epitope of proteins which subsequently are reproduced biologically or synthetically. Alternatively, peptidic ligands can be selected and produced through phage display techniques.

Another alternative for the selection of peptidic ligands are in particular epitope mapping and paratope mapping methods. These methods can be based on known sequence information of the proteins or antibodies in question, but also take place on the basis of combinatorial peptide libraries. Furthermore, the selection of the peptidic ligands can take place via computer-generated predictions or be supported by these.

Nucleic acid ligands can be selected by known methods (e.g., Selex, Conrad 1995).

The identified ligands are subsequently preferably produced synthetically and provided with a chemical function (linker) in the course of the synthesis, the chemical function permitting the integration and cross-linking with the structure-providing component during the production of the affinity binders. Known and, e.g., commercially obtained ligands may also later be provided with desired linker functions, if required. In the case of peptides and proteins and other organic molecules, known bioconjugation protocols using free thiol, carboxyl or amino functions, for example, can be used in this connection.

The linker function can in principle be each moiety capable of binding which allows for fixation of the ligands in the structure-providing component. This is typically a reactive moiety which can undergo a cross-linking reaction with an inorganic and/or organic cross-linker This cross-linking reaction is preferably a polymerization reaction, the term “polymerization” herein being understood as also including a polycondensation or polyaddition, and both the linker function and the cross-linker(s) bear polymerizable groups.

An inorganic cross-linker may be a bifunctional or multifunctional organosiloxane, for example.

In the case of an organic cross-linker, this may be an acyl derivative, methacryl derivative, e.g., ethylene glycol dimethacrylate or trimethylolpropane trimethacrylate, or allyl derivative, for example. To achieve a higher hydrophilicity of the polymer backbone, e.g., corresponding cross-linkers based on polyethylene glycol can be employed. Further suitable examples of cross-linkers are apparent to those skilled in the art without any difficulty.

In one example, the polymerization reaction only takes place between the linker moieties and one or more cross-linkers as well as optionally between the cross-linker molecules amongst each other. However, the polymerization reaction may also take place in the presence of further polymerizable monomeric, oligomeric or polymeric reaction partners which can undergo a polymerization reaction with the linker moieties and/or the cross-linker.

Suitable reaction partners for the respective linker functions or cross-linkers are known to those skilled in the art or can be determined easily by means of routine experimentation. The reaction partners may also be other polymerizable affinity binders, for example. The polymerization reaction can be a RAFT polymerization, for example.

By means of the polymerization reaction, a structure-providing component is formed which can comprise or essentially consist of an organic or inorganic polymer, e.g., a polyurethane, polyurea, polyester, polyamide, aminoplast, epoxy resin, silicones or copolymers or mixtures thereof (besides the integrated ligands).

Examples of suitable linker functions are, e.g., an acryl derivative, methacryl derivative such as ethylene glycol dimethacrylate or trimethylolpropane trimethacrylate, epoxides, isocyanates or an allyl derivative.

The cross-linking reaction, in particular, polymerization can take place as a free-radical polymerization, e.g., induced by light or thermally induced at sufficiently low temperatures to not affect the target substance, anionically or cationically.

When producing the affinity binders, the ligand is added to a template of the target substance such that the ligands can specifically attach to the target regions of the target substance. In the next step, these complexes are embedded in the structure-providing component and fixed therein, preferably cross-linked with this component as discussed above. The target substance is subsequently removed again such that a structure with a defined spatial arrangement of the ligands is formed.

This product has the capacity to specifically bind the respective target substance again, i.e., the now present affinity binder has the capacity to bind the target substance again selectively and with a defined strength.

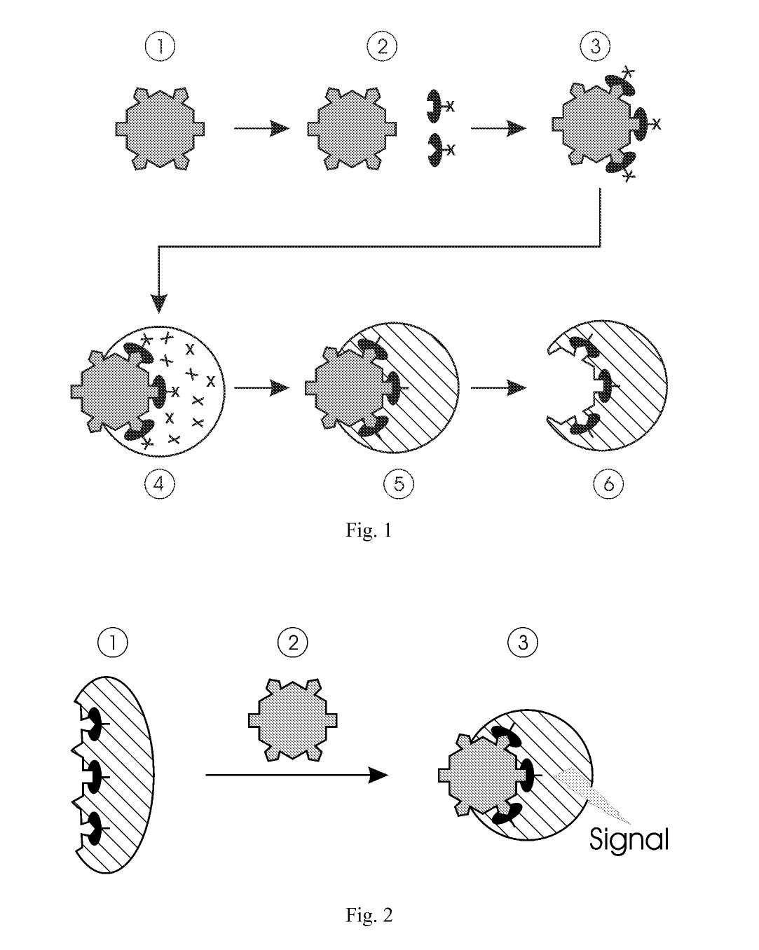

In an alternative example, the affinity binder is not—as depicted in FIGS. 1 and 2—a single body with a binding pocket, but a three-dimensional matrix with a plurality of binding points.

In a particularly preferred example of the affinity binders, these are provided with an effector function such that the binding process of the target substance is transferred into a visible or measurable signal or a signal characterized by other types (FIG. 2). Triggers for the effector function may be conformational changes, striction, charge transfer, reassociation, energy dissipation or similar mechanisms, for example. The effector function can be based on light activity (fluorescence, phosphorescence, chemiluminescence), color change, morphology change, electron transfer, charge separation and discharge or other principles.

An affinity binder which is obtainable by means of above method comprises a structure-providing component, for example, a polymer molecularly imprinted by a certain target substance, into which at least one affinitive ligand for the target substance, preferably several ligands are integrated.

The use of the affinity binders offers the following advantages:
By means of the combination of the structure-providing component which ensures a spatial induced fit of the target substance and at the same time supports the spatial arrangement of the integrated ligand(s) and the non-covalent molecular interaction of the target substance with the ligand(s), an effective affinity is achieved which results in a marked increase of the affinity in comparison to the respective individual components.
The affinity increases exponentially if—instead of one ligand—several ligands are integrated at defined positions of the structure-providing component which subsequently can interact with different domains of the target substance. In this connection, the ligands may be identical and react with a recurring structural feature of the target substance, but they can also be different ligands which only react with a particular (singular) structural feature of the target substance.
Through the cooperation of several ligands, an affinity (avidity) can be achieved which can be regulated stepwise and rationally and cannot be realized with conventional and, in particular, natural affinity binders. The affinity can be chosen in such a way that a non-destructive recovery of the target substance is possible (e.g., for separation/cleaning methods), but can also be fashioned irreversibly (e.g., for analytical methods aiming for highest sensitivity).
Both the structure-providing components and the ligands can be produced completely synthetically. For this reason, the affinity binders can be produced quicker and at a lower cost than proteinaceous binders (e.g., antibodies).
The affinity binders are chemically and physically markedly more stable than proteinaceous binders and may thus be employed under extreme environmental conditions.
The development and production of the affinity binders can in principle be designed independently from animal experiments and/or genetic engineering methods.
The affinity binders offer a breadth of usage options, for example, in (but not restricted to) medicine and diagnostics, analytics and bioanalytics, pharmaceutics, separation and cleaning methods, and can facilitate the production and handling in these fields and/or increase cost efficiency, versatility, stability, specificity and sensitivity of conventional methods.

The following examples shall explain our affinity binders and methods in further detail, but without limiting them to the details of these examples.

Our concepts can be applied to the production of an affinity binder (an affinity structure) for the binding of the high-molecular weight protein thyreoglobulin, for example. In clinical chemistry, thyreoglobulin is drawn upon as a tumor marker for thyroid carcinomas. The particularly sensitive detection of tumor markers can enable an earlier diagnosis of tumor events and improve the chances of curing the disease. Therefore, the affinity structure may be employed as the basis of a test system for an improved detection of thyreoglobulin.

### Example 1

### Identification and Production of Ligands for Thyreoglobulin

1. Paratope mapping of the monoclonal anti-thyreoglobulin antibody mAb Tg10 (procedure analogous to Daniel Laune et al., 2002)

Genetic analysis and nucleotide sequencing of the VH and VL regions: The genes of the light and heavy chains of the immunoglobulin G of the hybridoma cell line Tg10 were amplified by means of molecular biology using polymerase chain reaction and primer pairs which bind in the initial areas of the C_{H}1 and C_{L} regions and in conserved areas of the leader peptide sequence of the murine Ig gene family (Chardes et al., 1999). The nucleotide sequence of the amplificates were subsequently sequenced and translated into the corresponding amino acid sequences.

Paratope mapping: The amino acid sequence of the V_{H} and V_{L} domains identified above were substituted by overlapping peptides of 12 amino acids with an offset of an amino acid in each case. The peptides were synthetically produced and immobilized in an array format. The capacity of the peptides for the binding of the thyreoglobulin was tested by means of a labelled antigen in concentrations between 10⁻⁷ and 10⁻⁹ M. Peptide (A) having the amino acid sequence Ac-FTFGSG from the complementarity-determining region (CDR) L3 and peptide (B) having the amino acid sequence TFTSYGLTWVIQ from the CDR H1 of Tg10 were identified as suitable ligands.

### Example 2

### Production of an Affinity Binder for Human Thyreoglobulin

The production of the affinity binder (the affinity structures) took place by means of molecular imprinting of a polymer with integration of the peptidic ligands.

The peptides A and B identified in Example 1 were produced synthetically and provided with a spacer molecule (e.g., an alkyl or polyether unit) and a polymerizable group (linker) (e.g., an acryl derivative or methacryl derivative) in the course of the synthesis. The peptide synthesis took place on the solid phase according to the Fmoc strategy (Merrifield, 1963, Carpino and Hahn, 1970). In each case, one of the peptides in buffer solutions or buffer solutions mixed with organic solvents (MeOH, acetonitrile) are added to human thyreoglobulin in the ratio required in each case. The complexes of thyreoglobulin and peptide were dissolved/suspended in polymerizable cross-linker. The cross-linkers can be, e.g., ethylene glycol dimethacrylate or the trifunctional cross-linker TRIM (trimethylolpropane trimethacrylate). If a higher hydrophilicity of the polymer backbone is desired, corresponding polyethylene glycol-based cross-linkers are employed. Subsequently, cross-linking with the polymer induced by light or thermally at relatively low temperatures which do not affect the protein takes place. The thyreoglobulin used as the template is removed from the polymer such that a molecularly imprinted polymer with an integrated peptide ligand is obtained.

The affinity structures produced in this manner are suited to selectively bind and sensitively detect thyreoglobulin from a sample solution.

### Example 3

### Production of an Affinity Binder for Anti-Tumor Necrosis Factor-Alpha

Commercially available anti-tumor necrosis factor-alpha antibody (Santa Cruz Biotechnology Inc., Santa Cruz, Calif., USA) is proteolytically broken down into Fab fragments (“Fab Preparation Kit”, Pierce Biotechnology, Rockford, Ill., USA) which are capable to specifically bind tumor necrosis factor-alpha. The Fab fragments were provided with a polymerizable linker via an EDC/NHS bioconjugation protocol such as that described in Hermanson, 1996 and then contacted with the target substance tumor necrosis factor-alpha. The complexes of Fab fragments and the target substance were dissolved/suspended in polymerizable cross-linker (e.g., ethylene glycol dimethacrylate or TRIM) and polymerized analogously to Example 2. The tumor necrosis factor-alpha used as the template is removed from the polymer such that a molecularly imprinted polymer with integrated Fab fragments is obtained.

The affinity structures produced in this manner are suited to selectively bind and sensitively detect tumor necrosis factor-alpha from a sample solution.

1. A method for producing affinity binders having affinitive or highly affinitive binding structures for non-covalent interaction types, comprising:
a) contacting a target substance with at least one ligand which is capable of non-covalently binding to the target substance for a sufficient time that the at least one ligand can specifically attach to target regions of the target substance and form ligand-target substance complex;
b) embedding the complex in a structure-providing component and fixing by binding the ligand(s) of the complex to the structure-providing component; and
c) removing the target substance such that a structure with a defined spatial arrangement of the ligand(s) is formed which has capacity to specifically bind the respective target substance again.

2. The method according to claim 1, wherein several ligands are fixed and integrated in the structure-providing component.

3. The method according to claim 2, wherein the ligands are identical and interact with recurring motifs of the target substance.

4. The method according to claim 2, wherein the ligands are different and interact with different motifs of the target substance.

5. The method according to claim 1, wherein the at least one ligand is selected from the group consisting of a protein, peptide, carbohydrate, lectin, nucleic acid, low-molecular weight organic substance or mimetic of a protein ligand, peptide ligand, carbohydrate ligand and nucleic acid ligand.

6. The method according to claim 1, wherein the target substance is selected from the group consisting of a protein, peptide, carbohydrate, lectin, nucleic acid or another macromolecular unit, including a prokaryotic or eukaryotic cell, a virus and components thereof.

7. The method according to claim 1, wherein the structure providing component is or comprises an organic polymer.

8. The method according to claim 7, wherein the structure-providing component comprises a polyurethane, polyurea, polyester, polyamide, aminoplast, epoxy resin or copolymers of mixtures thereof.

9. The method according to claim 1, wherein the ligands are provided with a reactive, polymerizable linker.

10. The method according to claim 9, wherein the linker is selected from the group consisting of an acryl derivative, methacryl derivative, epoxide, and an isocyanate or allyl derivative.

11. The method according to claim 1, wherein the fixing in step b) includes cross-linking the ligand(s) with the structure-providing component.

12. The method according to claim 11, wherein the cross-linking takes place in the presence of an organic and/or inorganic cross-linker.

13. The method according to claim 12, wherein the cross-linker bears polymerizable groups.

14. The method according to claim 11, wherein the cross-linking reaction is a free-radical induced by light, or thermally induced, or anionic or cationic cross-linking reaction.

15. An affinity binder having affinitive or highly affinitive binding structures for non-covalent interaction types, obtained with the method according to claim 1 and comprising a structure-providing component and at least one ligand integrated into the structure-providing component, wherein the structure-providing component holds the at least one ligand in a certain spatial arrangement which permits or affects the specific binding of the ligand(s) to a target substance.

16. The affinity binder according to claim 15, wherein the specific spatial arrangement permits the binding of the at least one ligand to the target substance with an adjustable affinity.

17. The affinity binder according to claim 15, wherein the shape of the structure-providing component allows for a spatially induced fit of the target substance in the structure-providing component and thus can support binding of the target substance.

18. The affinity binder according to claim 15, possessing exactly one binding site for the target substance.

19. The affinity binder according to claim 16, which possesses several binding sites for the target substance.

20. The affinity binder according to claim 15, wherein the ligand is selected from the group consisting of a protein, peptide, carbohydrate, lectin, nucleic acid or mimetic of a protein ligand, peptide ligand, carbohydrate ligand and nucleic acid ligand.

21. The affinity binder according to claim 15, wherein several ligands are integrated into the structure-providing component.

22. The affinity binder according to claim 21, wherein the ligands are identical and interact with recurring motifs of the target substance.

23. The affinity binder according to claim 21, wherein the ligands are different and interact with different motifs of the target substance.

24. The affinity binder according to claim 15, wherein the target substance is selected from the group consisting of a protein, peptide, carbohydrate, lectin, nucleic acid, another macromolecular unit, a prokaryotic or eukaryotic cell, a virus and components thereof.

25. The affinity binder according to claim 15, wherein the structure-providing component is or comprises a molecularly imprinted polymer.

26. The affinity binder according to claim 15, wherein binding of the target substance is coupled with an effector function.

27. The affinity binder according to claim 26, wherein the effector function is selected from the group consisting of a conformational change, a striction, a charge transfer, a reassociation, an energy dissipation, a pH change, a conductivity change and a catalytic function.

28. (canceled)


SEQUENCE LISTING (2 sequences)

SEQ ID NO: 1 - protein, 6 aa, Artificial Sequence
  Description of Artificial Sequence Synthetic peptide
FTFGSG

SEQ ID NO: 2 - protein, 12 aa, Artificial Sequence
  Description of Artificial Sequence Synthetic peptide
TFTSYGLTWVIQ